(19) United States
(12) Patent Application Publication
(10) Pub. No.: US 2017/0266615 A1
(43) Pub. Date: Sep. 21, 2017

(54) GAS FILTERING APPARATUS FOR REDUCING ODOR

(71) Applicant: Hayim Eilat, Beit Shean (IL)

(72) Inventor: Hayim Eilat, Beit Shean (IL)

(21) Appl. No.: 15/458,336

(22) Filed: Mar. 14, 2017

Related U.S. Application Data
(60) Provisional application No. 62/310,745, filed on Mar. 20, 2016.

(51) Int. Cl. B01D 53/85; A61L 9/16

(52) U.S. Cl. CPC B01D 53/85; A61L 9/16; A61L 2209/14

(57) ABSTRACT

Disclosed is a gas filtering apparatus positioned in a closed environment for reducing odor from gases using compost mass. The gas filtering apparatus includes a first compartment, a second compartment and a layer. The first compartment is having an inlet for receiving gases and a perforated top surface. The first compartment allows the gases to move upwards through the perforated top surface. The second compartment is positioned on top of the first compartment. The second compartment includes a top section with an opening and an open bottom section. The second compartment stores organic compost for treating the received gases from the perforated top surface of the first compartment. The treated gas is released in the atmosphere through the opening. The layer is configured to attach to the open bottom section of the second compartment. The layer is configured to hold the compost mass in the second compartment. Further, the layer is allows the gases to pass from the perforated top surface of the first compartment to the second compartment.

### CROSS-REFERENCE TO RELATED APPLICATIONS

This application claims priority to U.S. Provisional Application No. 62/310,745 filed on Mar. 20, 2016, which is hereby incorporated herein by reference for all purposes.

### BACKGROUND OF THE INVENTION

1. Field of the Invention

The present invention generally relates to a gas filtering apparatus for reducing odor from closed environment and more particularly relates to a gas filtering apparatus for reducing odor using organic compost mass.

2. Description of Related Art

For recent years biological filtration is increasingly used for cleaning gas streams originating from various industrial activities like production processes, waste water transport and waste water treatment in order to reduce nuisance, trouble or damage to the environment caused by malodorant and/or poisonous components.

In view of the material often used for biological filtration, one also speaks of compost filtration. In biological filters other materials like peat, wood chips or fit-branches are used as the so called carrier materials. Wastewater treatment plants, rendering plants, food processing, flavor manufacturing and composting facilities generate significant volumes of waste gas streams containing odor causing compounds.

Prior to exhausting these waste gas streams into the environment, these facilities are required to treat the contaminated air to remove odor causing compounds such as ammonia, hydrogen sulfide and volatile organic compounds (VOC). Many methods are known to filter gas to reducing the odor such as cotton based filtering, plastic based filtering, coal based filtering, lave rocks filtering and water based filtering.

However, the current methods are using non-renewable sources and thus resulting in expensive material costs. Therefore, there is a need of a gas filtering apparatus for reducing odor created by ammonia and other chemicals using a combination of humidity and the bacteria in the compost.

### SUMMARY OF THE INVENTION

In accordance with teachings of present invention, a gas filtering apparatus positioned in a closed environment for reducing odor from gases using compost mass is provided.

An object of the present invention is to provide a gas filtering apparatus including a first compartment, a second compartment and a layer. The first compartment is having an inlet for receiving gases and a perforated top surface. The first compartment allows the gases to move upwards through the perforated top surface.

The second compartment is positioned on top of the first compartment. The second compartment includes a top section with an opening and an open bottom section. The second compartment stores organic compost for treating the received gases from the perforated top surface of the first compartment. The treated gas is released in the atmosphere through the opening.

The layer is configured to attach to the open bottom section of the second compartment. The layer is configured to hold the compost mass in the second compartment. Further, the layer is allows the gases to pass from the perforated top surface of the first compartment to the second compartment.

Another object of the present invention is to provide the gas filtering apparatus including at least one first pipe configured to pass through the second compartment to attach to the inlet of the first compartment. The first pipe transfers the gas from outside to the first compartment.

Another object of the present invention is to provide the gas filtering apparatus including at least one second pipe configured to attach to the inlet of the first compartment. The second pipe transfers the gas from outside to the first compartment. Further, the gas filtering apparatus includes a first pump to pump the gas from outside into the first compartment through the inlet.

Another object of the present invention is to provide the gas filtering apparatus including a second pump to pump the gas from outside into the first pipe. Further, the gas filtering apparatus includes a first pump to pump the gas from outside into the second pipe.

These and other features and advantages will become apparent from the following detailed description of illustrative embodiments thereof, which is to be read in connection with the accompanying drawings.

### BRIEF DESCRIPTION OF DRAWING

The disclosure will provide details in the following description of preferred embodiments with reference to the following figures wherein:

FIG. 1 illustrates a schematic diagram of a gas filtering apparatus in accordance with a preferred embodiment of the present invention; and

FIG. 2 illustrates another schematic diagram of a gas filtering apparatus in accordance with another preferred embodiment of the present invention.

### DETAILED DESCRIPTION OF DRAWINGS

While this technology is illustrated and described in a preferred embodiment, a gas filtering apparatus positioned in a closed environment for reducing odor from gases using compost mass may be produced in many different shapes, sizes, materials, forms and configurations. There is depicted in the drawings, and will herein be described in detail, as a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the technology described herein.

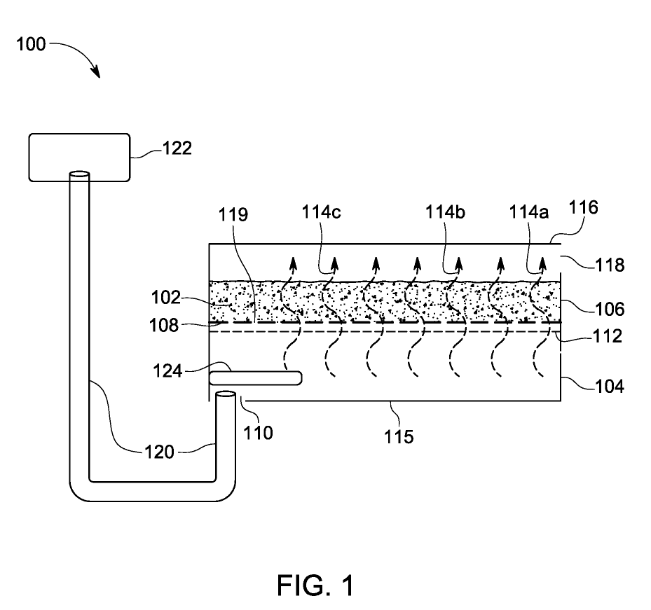

FIG. 1 illustrates a schematic diagram of a gas filtering apparatus 100 positioned in a close environment for reducing odor from gases using compost mass 102 in accordance with a preferred embodiment of the present invention. The gas filtering apparatus 100 includes a first compartment 104, a second compartment 106 and a layer 108. The bio compost mass is made up of fibers and active bacteria.

The first compartment 104 includes an inlet 110 for receiving gases and a perforated top surface 112. The first compartment 104 allows the gases to move upwards through the perforated top surface 112. The upward movement of gases is shown through plurality of arrows such as 114a, 114b and 114c.

Further in a preferred embodiment of the present invention, the inlet 110 is configured on bottom surface 115. The second compartment 106 is positioned on top of the first compartment 104. The second compartment 106 includes a top section 116 with an opening 118 and an open bottom section 119.

The second compartment 106 stores organic compost 102 for treating gases received from the first compartment 104 through the perforated top surface 112. Further, the treated gas is released in the atmosphere through the opening 118. The size of opening 118 may vary as per the requirements to treat the gases.

In a preferred embodiment of the present invention, the first compartment 104 with perforated top surface 112 and the second compartment 106 is rectangular in shape. However, those skilled in the art may envision various shapes of the first compartment 104 and the second compartment 106 without deviating from the scope of the present invention.

The layer 108 is configured in the second compartment to hold the organic compost mass in the second compartment. Further, the layer 108 allows the gases to pass from the perforated top surface 112 of the first compartment to the second compartment. The treated gas released in the atmosphere is odor free.

In a preferred embodiment of the present invention, the layer 108 is made up of foam. However, it would be readily apparent to those skilled in the art that various materials may be used to create the layer 108 without deviating from the scope of the present invention.

In another preferred embodiment of the present invention, the gas filtering apparatus 100 includes at least one second pipe 120 configured to attach to the inlet 110 of the first compartment 104. The second pipe 120 transfers the gas from outside to the first compartment 104.

In another preferred embodiment of the present invention, the gas filtering apparatus 100 includes a first pump 122 to pump the gas from outside into the second pipe 120. In a preferred embodiment of the present invention, the gas filtering apparatus treats harmful, odor gases with the biomass compost to release fresh and odor free gas in the atmosphere.

In another preferred embodiment of the present invention, the gas filtering apparatus 100 includes a filter 124 configured in proximity to the first pipe 120 to evenly distribute the gas flow throughout the entire first compartment 104. Examples of material filter 124 include but not limited to wood, metal, plastic, concrete, or any other material to prevent the gas flow upwards and distribute the gas flow throughout the entire first compartment 104, etc.

In exemplary embodiment of the present invention, the filter 124 is configured to either attach to sidewall of the first compartment 104 or configured like a chair over the first pipe 110 so as to prevent the gas flow upwards and distribute the gas flow throughout the entire first compartment 104.

Examples of material for creating the first compartment 104 and the second compartment 106 includes but not limited to metal, concrete, fiber glass, any other material that does not allow to pass the air etc. It would be readily apparent to those skilled in the art that various materials may be used to make the first compartment 104 and the second compartment 106 without deviating from the scope of the present invention.

In a preferred embodiment the material of the perforated top surface 112 is wood. However, it would be readily apparent to those skilled in the art that various materials may be envisioned for creating perforated top surface 112 that allows easy passage of gases, without deviating from the scope of the present invention.

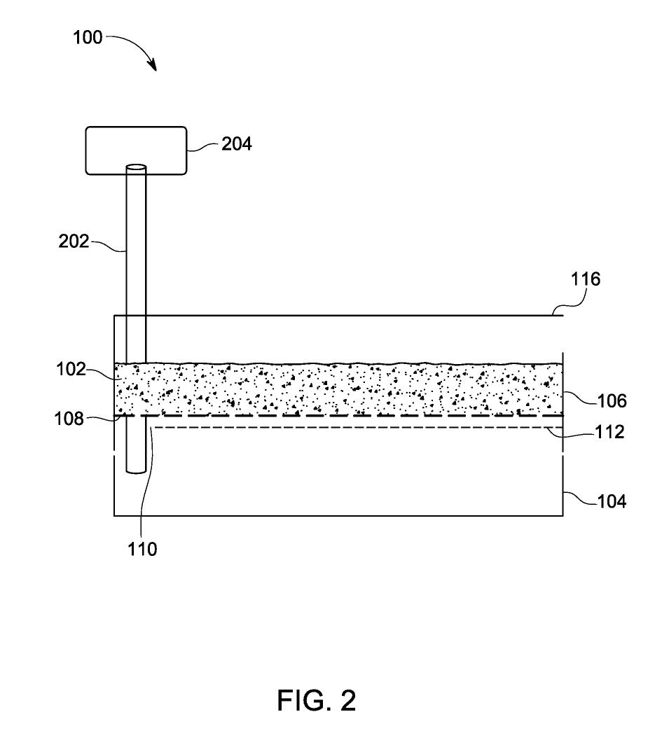

FIG. 2 illustrates another schematic diagram of a gas filtering apparatus 100 in accordance with another preferred embodiment of the present invention. In another preferred embodiment of the present invention, the inlet 110 is configured on the perforated top surface 112.

The gas filtering apparatus 100 includes a first pipe 202 configured to pass through the second compartment 106 to attach to the inlet 110 of the first compartment 104. The first pipe 202 transfers the gas from outside to the first compartment 104. Further, the gas filtering apparatus 100 includes a second pump 204 to pump the gas from outside into the first pipe 202.

The present invention offer various advantages such as high availability of the of the components such as wood and compost of the bio filter along with low cost. Moreover, due to the moisture in the compost mass, the preset invention is able to filter out odor more effectively than other types of bio filters.

Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

1. A gas filtering apparatus positioned in a closed environment for reducing odor from gases using compost mass, comprising:
a first compartment having an inlet for receiving gases and a perforated top surface, further the first compartment allows the gases to move upwards through the perforated top surface;
a second compartment positioned on top of the first compartment, the second compartment comprising a top section with an opening and an open bottom section, wherein the second compartment stores organic compost for treating gases received from the first compartment through the perforated top surface, further the treated gas releases in the atmosphere through the opening; and
a layer configured in the second compartment to hold the organic compost mass in the second compartment and further the layer allows the gases to pass from the perforated top surface of the first compartment to the second compartment.

2. The gas filtering apparatus according to claim 1 further comprising at least one first pipe configured to pass through the second compartment to attach to the inlet of the first compartment, the first pipe transfers the gas from outside to the first compartment.

3. The gas filtering apparatus according to claim 1 further comprising at least one second pipe configured to attach to the inlet of the first compartment, further the second pipe transfers the gas from outside to the first compartment.

4. The gas filtering apparatus according to claim 3 further comprising a first pump to pump the gas from outside into the second pipe.

5. The gas filtering apparatus according to claim 2 further comprising a second pump to pump the gas from outside into the first pipe.

6. The gas filtering apparatus according to claim 2 further comprising a filter configured in proximity to the first pipe to evenly distribute the gas flow throughout the entire first compartment.
